(19) United States
(12) Patent Application Publication
(10) Pub. No.: US 2016/0144100 A1
(43) Pub. Date: May 26, 2016

(54) MICRO-NEEDLE DRUG DELIVERY SYSTEMS

(71) Applicant: CALIFORNIA INSTITUTE OF TECHNOLOGY, Pasadena, CA (US)

(72) Inventors: Morteza Gharib, Altadena, CA (US); Julia Cosse, Pasadena, CA (US); Stephanie Rider, Monrovia, CA (US); Cong Wang, Pasadena, CA (US)

(21) Appl. No.: 14/947,767

(22) Filed: Nov. 20, 2015

Related U.S. Application Data
(60) Provisional application No. 62/083,070, filed on Nov. 21, 2014.

(51) Int. Cl. A61M 5/148; A61M 37/00

(52) U.S. Cl. CPC A61M 5/148; A61M 37/0015; A61M 2037/003; A61M 2037/0061; A61M 2037/0023

(57) ABSTRACT

Generally, devices, systems and methods of using one or more needle(s) or micro-needle arrays are described in which a shift of fluid volume from a first expansion member position to a second expansion member position drives delivery of the fluid, which can be a drug or drug solution, through the needle(s) or micro-needle arrays.

### RELATED APPLICATIONS

This filing claims the benefit of and priority to U.S. Provisional Patent Application No. 62/083,070 filed Nov. 21, 2014, which is incorporated by reference herein in its entirety for any and all purposes.

### FIELD

This filing relates to micro- and/or nano-scale needle (hereinafter “microneedle”) and microneedle array type devices, and more particularly to associated fluid delivery features for forcing liquids (often drugs) through the needle(s) for effective intradermal, subcutaneous or other drug injection.

### BACKGROUND

In the last few decades, the development of new drugs for human use with improved potency has been a focus in the pharmaceutical field. However, the administration of such drugs has been limited due to poor absorption and enzymatic degradation in the gastrointestinal tract as well as painful delivery using intravascular injection.

Microneedle delivery systems offer an attractive option for administering those drugs across the skin. Microneedle use is promising because (typically in an array) such structures can provide holes to bypass the stratum corneum of skin for drug delivery with little or no pain.

The type of microneedles used may be categorized as luminal or dissolvable. Dissolvable microneedles include a polymer tip that dissolves when in contact with body fluid to deliver a drug, vaccine inoculation or other therapeutic agent. As the designation implies, luminal microneedles are bodies that include a lumen therein. The lumen in this class of microneedle may be used to deliver compounds in connection with various reservoir means such as described in U.S. Pat. No. 3,964,482 or U.S. Pat. No. 8,257,324.

Microneedles are sometimes made from stainless steel or other metals; other microneedles are fabricated employing micro-replication techniques, such as by injection molding of plastic material. The resulting products often fail to offer many of the advantages associated with microneedles and microneedle arrays fabricated from carbon nanotubes (CNTs). CNT-based microneedles have unmatched advantages due to their exceptional mechanical properties and simple fabrication processes. Irrespective of the type of luminal micro-needles to be used herein, improvement to the fluid handling hardware feeding the needle array may be of great value as further described.

### SUMMARY

Micro-needle penetration depth limits associated drug delivery rate and volume. The subject devices, systems and methods involve an injection approach that can generate high liquid speed to increase penetration depth. Another possible advantage is to enable maintaining a high pressure (as discussed further below) through drug (or other liquid) discharge.

Yet another possible advantage is to provide a delay action between the application of pressure by finger and onset of fluid discharge. Still another possible advantage is in providing a system that operates automatically (e.g., for needle insertion and fluid delivery therefrom in the same sense of a device that does once a latch is released) without the sound and/or associated physical force feedback (as in a jerk or rattle) after push-button actuation. These advantages (i.e., delay and/or so-called “automatic” action) may offer a use and/or user adoption advantage(s) in physiologically decoupling a physical actuation (e.g., finger squeeze and depression) from any associated discomfort. As such, a user may not “jump” or “flinch” such as when performing a manual or spring-loaded finger stick procedure. Avoiding such action may help inadvertent disruption of delivery device position and/or microneedle position or engagement. Other related use benefits may be observed as well.

Generally, needle and micro-needle array devices are described in which a shift of fluid volume from a first expansion member position, such as a first balloon, to a second expansion member position, such as a second balloon, drives fluid (often drug) delivery through a lumen of (each) of the needle(s). A single tube may define the balloon sub-system. It may be variously configured as detailed below or otherwise. Devices, overall systems and associated methods of use are also detailed.

The subject devices or systems (filled or unfilled with fluid including any drug), kits in which they are included (with and without assembly), methods of use and/or manufacture are all included within the scope of the present disclosure. Some aspects of the same are described above, and more detailed discussion is presented in connection with the figures below. Other devices, systems, methods, features and advantages of the subject matter described herein will be or will become apparent to one with skill in the art upon examination of the following figures and Detailed Description. It is intended that all such additional devices, systems, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE DRAWINGS

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely. The figures in the form of mechanical drawings or CAD models may, however, provide antecedent basis for any size, shape or relative size or shape ratio limitation that may be claimed.

FIGS. 1A and 1B are side and top views, respectively, of a device or system embodiment hereof;

FIG. 2 is an assembly view of the embodiment in FIGS. 1A and 1B;

FIG. 3 is an action-shot of the same embodiment ejecting fluid.

FIG. 4 is a perspective view of another device or system embodiment;

FIG. 5 is a semi-transparent side-section view of the FIG. 4 embodiment being actuated by a user.

FIG. 6 is a diagram of a balloon-system model;

FIG. 7 is a diagram of the model in FIG. 5 as employed in device or system embodiments hereof.

FIG. 8A is a graph of expected performance of balloon inflation; FIG. 8B is a graph of actual balloon performance in embodiments hereof.

FIG. 9 is a graph that shows synchronicity between pressure and radius over time for the subject output balloon.

FIG. 10 is a graph that shows pressure oscillation over time between different balloons embodiments.

FIG. 11 is a flowchart detailing methods of embodiment use.

### DETAILED DESCRIPTION

Various exemplary embodiments are described below. Reference is made to these examples in a non-limiting sense, as it should be noted that they are provided to illustrate more broadly applicable aspects of the devices, systems and methods. Various changes may be made to these embodiments and equivalents may be substituted without departing from the true spirit and scope of the various embodiments. In addition, many modifications may be made to adapt a particular situation, material, composition of matter, process, process act(s) or step(s) to the objective(s), spirit or scope of the present invention. All such modifications are intended to be within the scope of the claims made herein.

Before the present subject matter is described in detail, it is to be understood that this disclosure is not limited to the particular example embodiments described, as such may, of course, vary. It is also to be understood that the terminology used herein is for the purpose of describing particular embodiments only, and is not intended to be limiting, since the scope of the present disclosure will be limited only by the appended claims.

All features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Express recitation of every possible combination and substitution is overly burdensome, especially given that the permissibility of each and every such combination and substitution will be readily recognized by those of ordinary skill in the art upon reading this description.

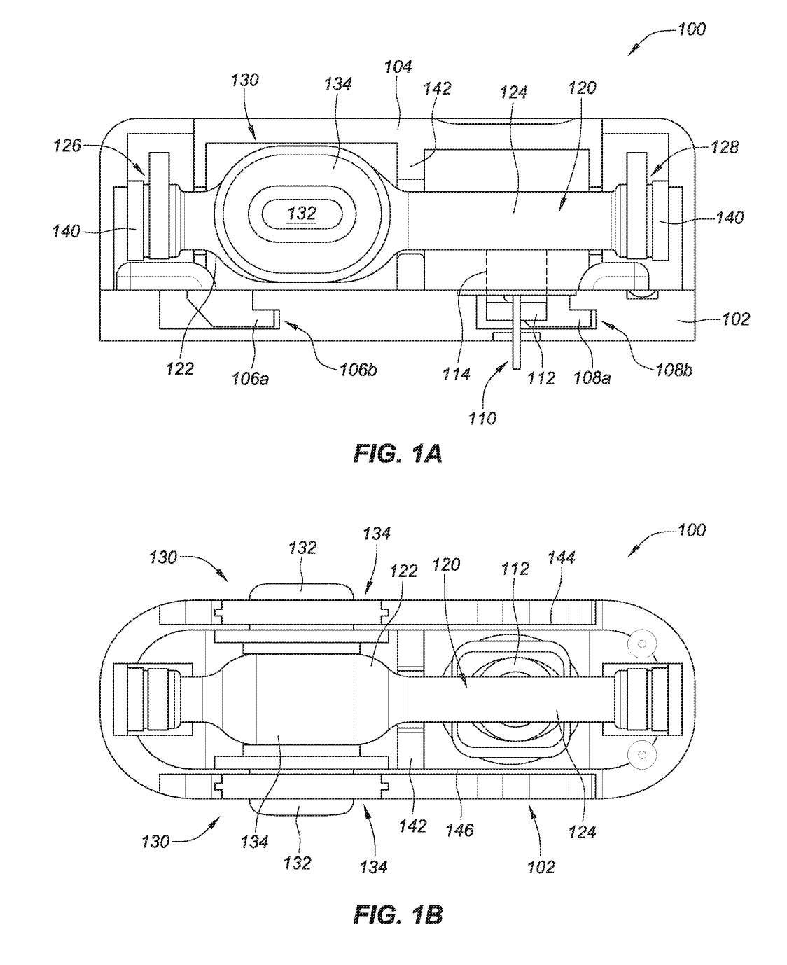

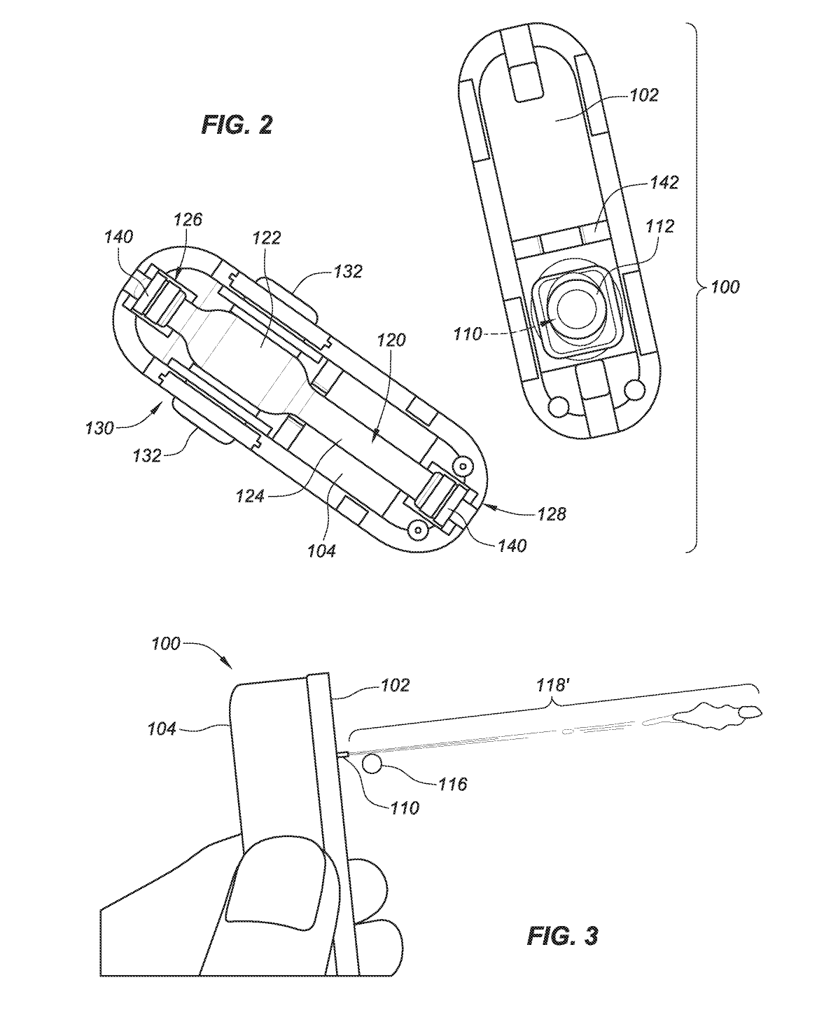

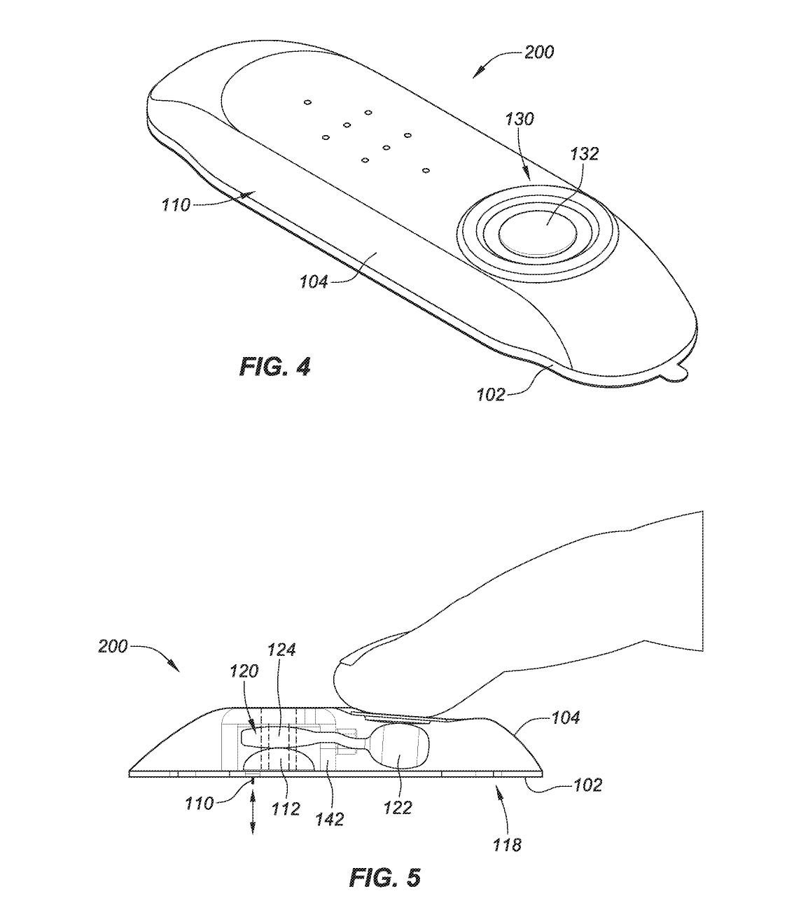

Two hardware embodiments are first described. An overview of a first injection or injector device embodiment 100 is shown in FIGS. 1A and 1B with details of construction and operation in FIGS. 2 and 3, respectively. An overview of a second injection or injector device embodiment 200 is shown in FIG. 4 with construction and operation details shown in FIG. 5.

Regarding injector device 100, it includes a body member, in this case with upper and lower portions (e.g., as a base or bottom plate 102 and cover 104). These are connected by optional interface features 106a, 106b and 108a, 108b. Various other snap or press fit features may be employed as may be gluing, chemical or ultrasonic welding, press-fitting or other approaches. These approaches may offer a permanent connection such that all of the resulting device is intended for single use as a disposable. Alternatively, the base or bottom plate 102 and associated components may be set for single use and the top applicator portion (associated with cover 104 or otherwise) reusable base members, cartridges, etc.

In any case, the lower body portion includes base plate 102 and a needle or needle array 110 extending therefrom. The needle(s) are shown in fluid communication with a reservoir 112 held by base plate 102.

Fluid (often an aqueous drug solution) is driven through the needle(s) from reservoir 112 when an adjacent tube 120 section is expanded. A proximal tube portion 122 is shown in an expanded or pre-expanded state defining a first volume. By side-to-side compression at user interface region(s) 130—specifically, finger-depressible elastic buttons 132 filing or occupying windows 134—fluid content at the proximal tube portion 122 is shifted to a distal tube portion 124 (as indicated by dashed line).

Both a proximal end 126 and a distal end 128 of the tube 120 are closed. Plugs 140 may be employed for such purpose or other means (such as sealing by adhesive, welding, etc.).

Other optional device or system features include a guide 142 separating the proximal and distal sections of tube 120 so that discrete fluid volume transfer occurs. Stated otherwise, guide 142 ensures the separation of a first (pre-actuation) volume and first “balloon” position from a second (post-actuation) volume and second balloon position in or of the tube. Other guide features such as body walls 144, 146 may further (or otherwise) constrain volume expansion

Another option is for direct connection of reservoir 112 with tube 120 (as indicated by dashed line connection 114) or other integration of the bodies. Without such connection (or integration) the bodies are separate or separable such as shown in FIG. 2 or otherwise.

As with distal section 124, the entire tube 120 may be substantially cylindrical. Or the proximal tube section 122 may be expanded or bulbous in shape as in a so-called “balloon” shape as an expansion member.

As shown in FIG. 3, user finger compression or button depression at the interface region(s) 130 results in expelling fluid 116 as a pressurized jet 116′ from the needles or needle array 110. The needle(s) may be fabricated in any manner as discussed above or otherwise. However, they advantageously comprise CNT microneedles provided in an array. Suitable examples of structures and associated fabrication techniques are provide in U.S. Pat. No. 7,955,644 and USPPNs 2010/0196446, 2011/0250376, 2012/0021164 and 2012/0058170 each of which document incorporated herein by reference in its entirety.

Such a jet or expulsion of fluid is advantageously characterized by its speed. For example, the expelled or expulsed fluid may vary in speed from about 2 m/s to about 10 m/s and up to about 20 m/s or more by employing different (e.g., thicker and/or higher modulus) tube material selection for greater force or impulse load transfer along with the moving fluid volume. Selection of liquid or high-pressure gas for the fluid may similarly assist in achieving higher jet speeds for injection. For gas as the fluid within tube 120, it may be set to a relatively high internal pressure (e.g., at least 20 psi and upwards of 50 psi, so about 1.5 atm to about 3.5 atm, or more such as up to 60 psi or 4 atm pressure); when provided at such pressures, gasses will transfer along tube 120 faster and/or provide as stiffer, stronger or higher spring rate within the tube. And while liquid may transfer along tube slower, is relatively greater mass can provide greater impulse loading to drive drug jet injection. Furthermore, the incompressibility of the liquid eliminates any spring rate associated with a gas-filled chambers in terms of a so-called “air spring” irrespective of gas composition. Duration of injection flow will vary with the above depending on the volume of liquid in the liquid reservoir and administration pressure.

FIG. 4 illustrates another injector device embodiment 200 that may operate similarly. Likewise, similar elements are provided (e.g., as sharing their callout numbers in addition to what is described below), but the design is configured for one finger operation. FIG. 5 illustrates index finger depression of single button 132 to actuate the device.

As with embodiment 100, embodiment 200 in FIGS. 4 and 5 is configured with a body and a needle or needle array (e.g., a micro-needle array) extending therefrom for insertion into the skin of a subject or user. With both embodiments, a base or lower surface 118 of the device is laid against the subject's skin and the needle(s) manually driven therein for fluid injection.

However, it is contemplated that action of the distal tube portion 124, which expands after proximal tube portion is compressed, may instead drive the needle(s). Such action would be facilitated by a spring-loaded slide or carriage as indicated by double-arrow in the FIG. 5. The same approach may be applied in or with embodiment 100 as well.

Regardless, a highly ergonomic and user-friendly injection device or system is provided in which depression of one or more buttons or other user-interface means shifts fluid in a tube 120 from a (proximal) first volume position to a (distal) second to drive fluid (from the tube and/or a reservoir 112) for injection without an abrupt impulse force (such as a tick, click or spring-loaded latch release) noticeable by a user. Nevertheless, the injection pressure and associated injection flow is sustained as further described below.

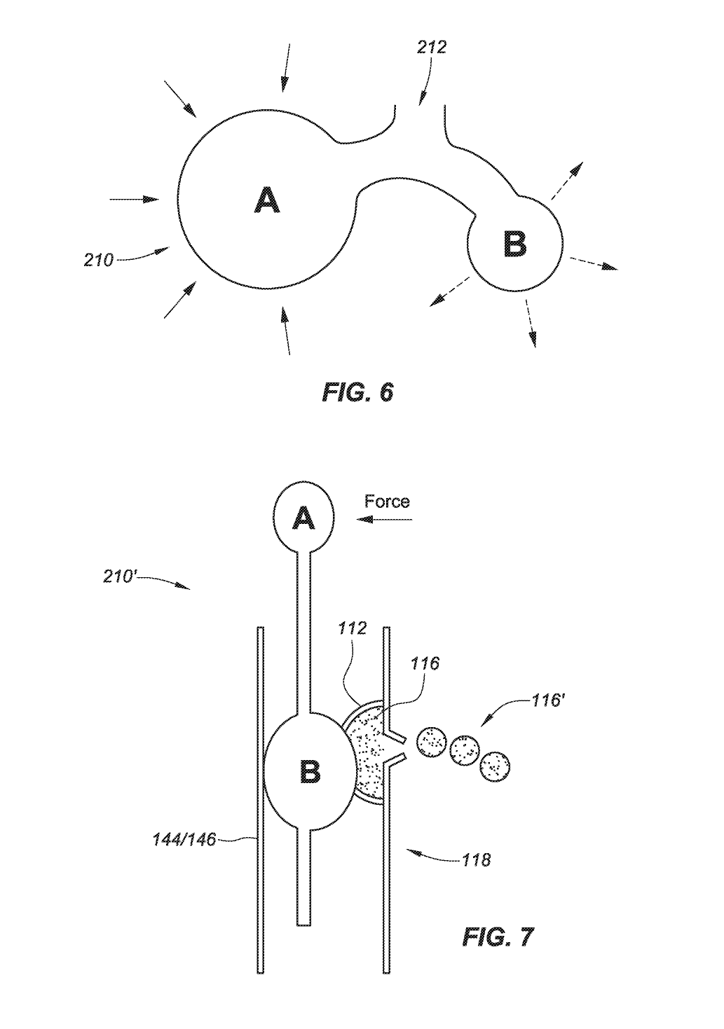

The subject approach can be modeled as involving two connected elastic balloons (A and B) in a system 210 filled with fluid (be it gas, optionally at a pressure to limit its compressibility or increase its spring rate, or generally incompressible liquid) as illustrated in FIG. 6.

Initially, the system is inflated with balloons A and B filled to the degree shown via port 212 or otherwise. Upon sealing the system, balloon B remains minimally inflated until pressure in balloon A reaches a threshold by force supplied thereto by a user through compression action of a finger or hand or otherwise mechanically. Such action is indicated by solid arrows. Balloon B then experiences sudden expansion as indicated by dashed arrows. For gas filled balloons, the delay action (i.e., a delay of expansion of balloon B) is experienced—in part—due to gas compressibility. Upon compressing balloon A, the pressure of gas inside does not immediately increase but instead, the increase will lag the mechanical compression. Another reason for the delay is because the compression does not immediately increase the inner pressure to the critical value to bulge balloon B. Thus, for liquid filling, the delay is weaker (i.e., shorter) as compressibility is negligible, but the time delay to build up pressure is still present.

The so-called “Two-Balloon Problem” (Laplace Law) explains such action. FIG. 7 diagrams such a result. In this system 210′, balloon A is decreased in size as fluid volume therein has been shifted or transferred to balloon B. With balloon B adjacent or otherwise in contact with a reservoir 112 of drug solution 116, the pressure now maintained by balloon B drives the fluid (again, typically a drug solution) therefrom. As such, high pressure in balloon B after the impulse (propagated as a pressure wave in the fluid) transferred from balloon A maintains a high delivery speed of the injection fluid. Moreover, the temporal delay associated with the build-up of pressure to accomplish volume shift from balloon A to balloon B avoids mistaken trigger and improves safety in operation.

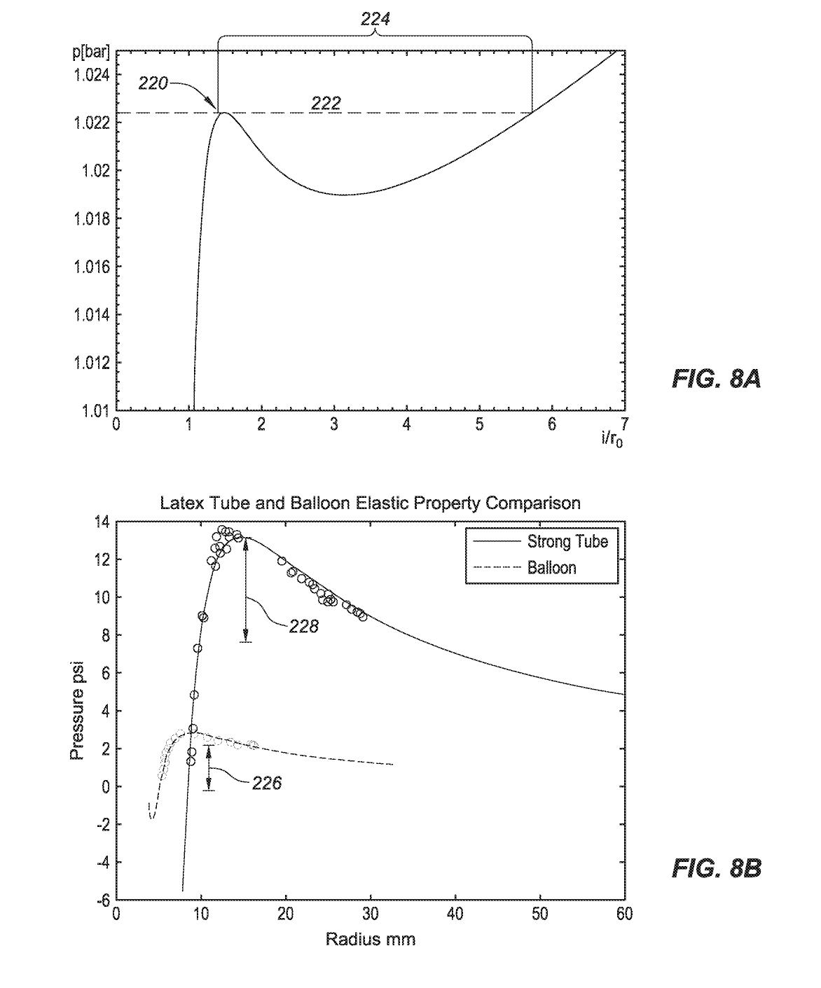

FIG. 8A is a graph of expected performance of balloon inflation, as would occur in balloon B of the two balloon system of FIG. 7. An elastic material (particularly a Mooney-Rivlin material) balloon behaves as plotted in FIG. 8A following the formula:

$$[ P ] ( r ) = 2 {S}_{1} \frac{d}{R} ( \frac{R}{r} - {( \frac{R}{r} )}^{7} ) ( 1 - \frac{{S}_{1}}{{S}_{2}} {( \frac{r}{R} )}^{2} )$$

where S₁ and S₂ are constants determined for the material, d is balloon thickness, r is balloon radius as it changes and R is balloon radius in relaxed state. This curve is characterized by a peak or spike 220. After reaching this threshold value 222, a region of expansion 224 is experienced by the balloon, even at lower pressure, until the pressure required for further filling climbs again thereafter. Before the pressure reaches the threshold value 222, balloon radius also increases somewhat, but pressure builds up. After this critical pressure is reached, the balloon radius expands faster but pressure actually drops. Accompanying this pressure drop (as is expected to occur in balloon B), a strong impulse (as pressure expected in balloon A is still high) is delivered by balloon B expansion against reservoir 112 for driving fluid (e.g., medicinal fluid) therefrom. Due to the mass of the liquid in the tube (if liquid is selected for the fluid within the tube of the two balloon system), a higher impulse is expected accordingly.

FIG. 8B is a graph of actual performance of balloon elements that may be used in the subject embodiments. With a basic balloon (i.e., a commercially available latex balloon with constants S₁=644 and S₂=367), little pressure and a smaller threshold pressure difference 226 between initial filling and further expansion is observed. A stronger tube (i.e., with material constants S₁=1918 and S₂=419) used for the balloon(s) shows a greater difference 228. Accordingly, a relatively stronger balloon material may be preferred for tube 120 in some embodiments. So-selected, when volume transfer occurs from balloon or balloon section A to balloon or balloon section B, the latter will fill to greater degree, essentially evacuating the former.

It may also be useful to select a tube material that is highly thermally insulative or insulated so as to avoid any temperature-related decrease of pressure over time associated with a gas (e.g., as related per the Ideal Gas Law) as further explained below. Alternatively, selection of liquid for the fluid internal to the tube can mitigate or altogether avoid such temperature-pressure type effects since very little volume change is experience with temperature variation of many liquids.

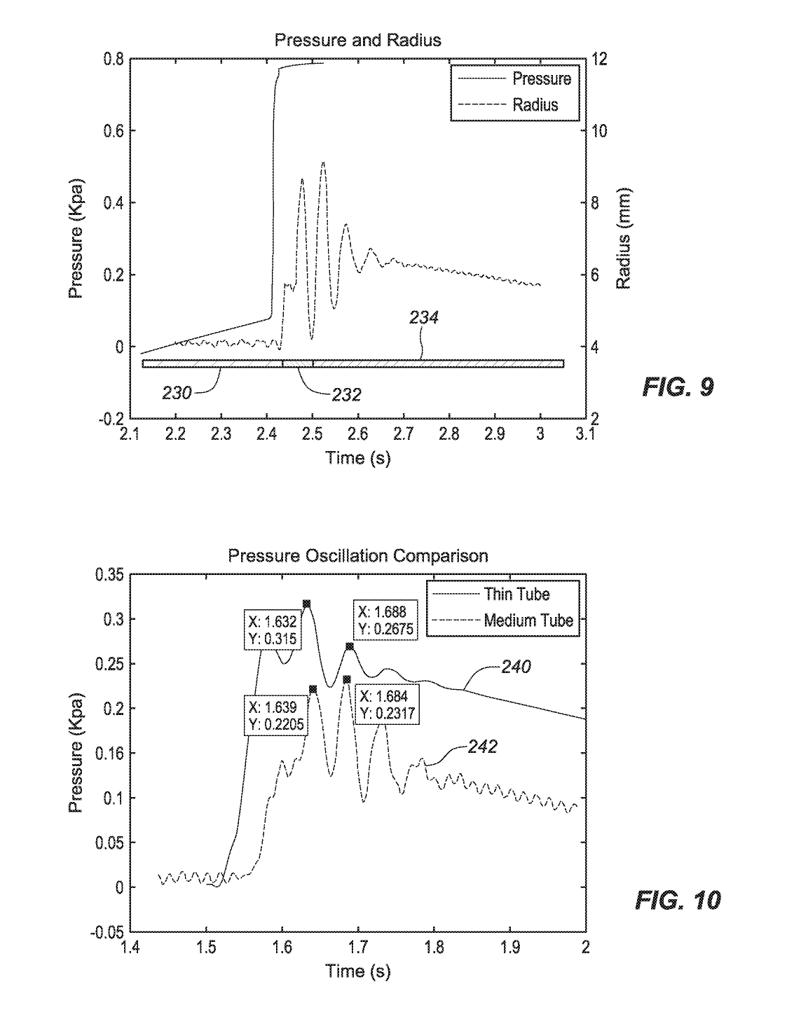

FIG. 9 is a graph that demonstrates the synchronicity between pressure and radius for the subject output balloon (i.e., balloon B in the model above and tube portion or sections 124 in each of embodiments 100 and 200). The y-axis scale at the left of the graph shows balloon radius in millimeters, the y-axis scale at the right shows balloon pressure in kilopascals, and the x-axis scale at the bottom of the graph shows time in seconds from before an actuating event (at the left) until after (at the right). In a first phase 230, corresponding to a user squeezing the input balloon (i.e., balloon A in the model above and tube section 122 in each of embodiments 100 and 200) little diameter and/or pressure change is noted. Then in a second phase 232, a peak pressure and maximum radius is achieved as fluid volume transfer from balloon A to balloon B occurs, along with oscillations in pressure caused by balloon B suddenly expanding with an associated pressure wave. This wave oscillates back and forth inside the tube, with the wave magnitude decreasing as the kinetic energy is transferred to internal (thermal) energy bringing balloon internal pressure up according to Ideal Gas Law (i.e., where ΔP∝ΔT). After balloon B stabilizes, its internal pressure starts to decrease linearly in a third phase 234 when heat is lost from the air (in this example) inside the tube or balloon. As such, FIG. 9 illustrates the actuated balloon's ability to maintain pressure and perhaps to maintain it better when the actuated balloon is better insulated.

In use, when the fluid or “bubble” is shifted from the actuating balloon (i.e., balloon A in the model) to the actuated balloon (i.e., balloon B in the model), the fluid will remain shifted to provide force in connection with the actuated balloon (i.e., balloon B) on the drug reservoir (or as the drug reservoir) until all drug has been released. The so-called bubble does not shift back to its original position without manual actuation. As shown in FIG. 9, a time duration of each phase 230, 232 and 234 of an actuating event is evident along the x-axis of the graph. In the example embodiment, a sustained pressure for a drug (or other fluid) injection using the needle(s) is associated with the transferred bubble in balloon B and is available for about 0.5 seconds or more. Similar timeframe and pressure relationships are shown in the graph of FIG. 10 for a balloon or balloon sections inflated in the manner described above.

In FIG. 10, two time-pressure curves are shown. First is a time-pressure curve 240 for a balloon made from a smaller diameter tube, while second is a time-pressure curve 242 for a balloon made from a larger diameter tube. Both tubes shown as tested had the same wall thickness. Comparison of time-pressure curves 240 and 242 yields some interesting results. First, the periodicity of the oscillations is remarkably similar between the curves, indicating that this oscillation is likely caused by a pressure wave inside the tube and shown to be traveling at about the speed of sound from balloon A to balloon B. Also notable is a similar linear decrease in pressure over time (e.g., after the 1.8 second mark for each curve), which is likely a temperature-related phenomena (as described previously) that can be mitigated by tube (and/or overall device) insulation and/or tube or balloon fluid selection.

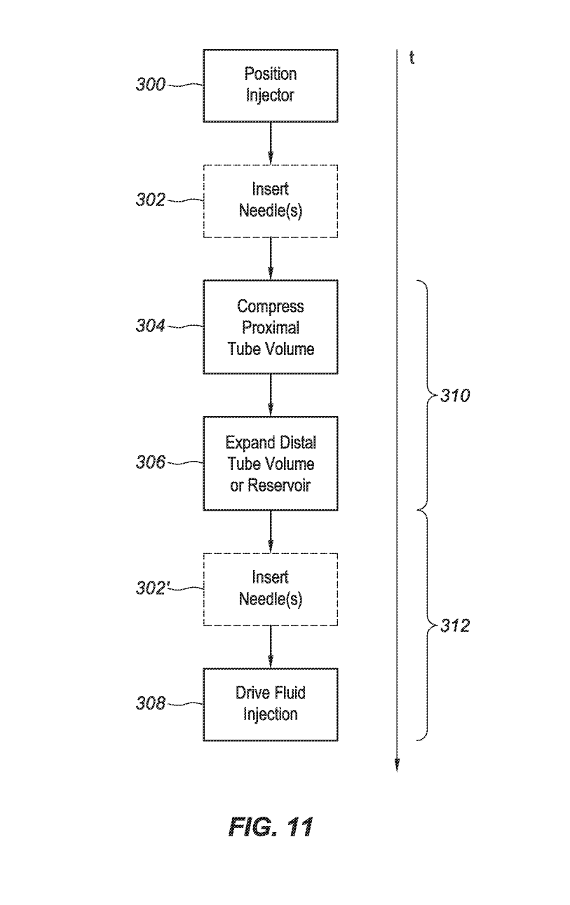

FIG. 11 is a flowchart detailing method steps possible with the subject devices and/or systems, according to various embodiments. At 300, an injector device or system is initially positioned against the skin of a subject. This step may be performed personally by a subject user to self-administer treatment or by another individual administering treatment but both will be referred to as the “user” below. The needle(s) may be inserted in connection with such positioning or by a separate downward pressure by the user at 302, or this may occur automatically later at 302′ as will be further described below. In many embodiments, at 304, a first volume of fluid (e.g., in tube 120 at proximal section 122) is compressed, causing the fluid to shift or otherwise be pushed or squeezed to a second volume position or location (e.g., in tube 120 at distal section 124) at 306.

Such action expands the distal tube volume and/or the fluid reservoir (depending on how the system is configured per example embodiments described above or otherwise) driving treatment fluid injection through the inserted needle to the application site at 308. Accordingly, one method embodiment may involve expelling treatment fluid from an externally pressurized or pushed-on reservoir. Another method may involve expelling treatment fluid directly from an expanded body or reservoir, where treatment fluid is stored in the tube itself. Other approaches (including combined activities) are possible as well. In some embodiments, the transfer or fluid or expansion action at 306 may also drive needle insertion into the skin to an application site at 302′ if not already performed.

As pictured against time axis (t) in FIG. 11, a shifting or transfer of fluid volume occurs over a delay period 310. This delay period may be controlled by tube fluid selection (i.e., as in choice between gas and liquid and/or selected gas pressure) and/or tubing material selection as it relates to a pressure build-up required for “balloon B” inflation. Such delay may have a duration from about 0.2 to about 2 or 3 seconds. To provide an adequate response time and associated tube 120 physical parameters, delay period 310 is preferably between about 0.5 and somewhere between about 1.0 to 1.5 seconds. In various embodiments, starting with expansion of a second volume (i.e., balloon B in the model above), treatment fluid delivery 308 occurs over a sustained period of time and/or pressure decay 312 as indicated (e.g., as described above as related to a drug volume to be delivered or administered by injection).

In some embodiments, the user, patient, subject or other individual performs device or system positioning and compression (such as manually with a hand, with a pair of fingers, with a finger and thumb or with a single finger). The method may further comprise the user pushing or inserting the needle or needle array into the skin at or near an application site. Needle insertion may also be performed automatically as a consequence of volume transfer or shift by user actuation as described previously. In any case, the treatment fluid delivered may comprise one or more drugs, medicaments or other substances.

The subject methods, including methods of use and/or manufacture of the hardware described, may be carried out in any order of the events which is logically possible, as well as any recited order of events. Furthermore, where a range of values is provided, it is understood that every intervening value, between the upper and lower limit of that range and any other stated or intervening value in the stated range is encompassed within the invention. Also, it is contemplated that any optional feature of the inventive variations described may be set forth and claimed independently, or in combination with any one or more of the features described herein.

Though the invention has been described in reference to several examples, optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention, where such changes or substitutions do not compromise the nature of the device or spirit of the concepts described herein.

Reference to a singular item includes the possibility that there are a plurality of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity. Accordingly, the breadth of the different inventive embodiments or aspects described herein is not to be limited to the examples provided and/or the subject specification, but rather only by the scope of the issued claim language.

1. An injection apparatus comprising:
a body member;
at least one needle extending from the body member and having a lumen in fluid communication with a reservoir;
an expansion member having elastic properties, being at least partially filled with a shiftable fluid and sealed at a proximal location, and being adjacent a user interface region;
wherein the user-interface region is configured to allow the expansion member to be compressed by operation of the user interface region in order to expand the expansion member at a distal location to drive a treatment fluid from the reservoir.

2. The apparatus of claim 1, wherein the expansion member adjacent the user-interface region is bulbous.

3. The apparatus of claim 1, wherein the expansion member is a cylindrical tube.

4. The apparatus of claim 1, wherein the expansion member and the reservoir are in fluid communication.

5. The apparatus of claim 1, wherein the distal location of the tube overlays the reservoir opposite the at least one needle.

6. The apparatus of claim 1, wherein the shiftable fluid and treatment fluid are a single fluid that is a drug solution.

7. The apparatus of claim 1, wherein the treatment fluid is a drug.

8. The apparatus of claim 1, wherein the expansion member is sealed at the distal location.

9. The apparatus of claim 1, wherein the user-interface region is selected from an open window and a depressible boss or button.

10. The apparatus of claim 1, wherein the body member comprises a base plate and top cover.

11. The apparatus of claim 10, wherein at least one of the base plate and top cover include at least one guide member to restrain expansion of the expansion member in at least one section of the body.

12. The apparatus of claim 11, wherein a distal side of the expansion member is not restrained from expanding.

13. The apparatus of claim 12, wherein the reservoir is a portion of the distal side of the tube.

14. The apparatus of claim 12, wherein the reservoir is not a portion of the distal side of the tube.

15. The apparatus of claim 1, wherein the at least one needle comprises an array of microneedles.

16. The apparatus of claim 15, wherein the array of microneedles comprises carbon nanotube microneedles.

17. A method of injecting fluid through a subject's skin to an administration site, the method comprising:
positioning an injector comprising at least one needle having a lumen and a reservoir against the skin;
compressing a proximal, first expansion member to expand a distal, second expansion member; and
flowing a treatment fluid through the at least one needle using pressure maintained in the second expansion member.

18. The method of claim 17, further comprising pushing the at least one needle into the skin.

19. The method of claim 17, wherein the subject performs the method.

20. The method of claim 17, wherein the at least one needle comprises an array of microneedles.

21. The method of claim 17, wherein the treatment fluid comprises a drug.

22. The method of claim 17, wherein the first and second expansion members are fluidly coupled by a tube.

23. The method of claim 17, wherein expansion of the second expansion member compresses a reservoir for the treatment fluid.

24. The method of claim 17, wherein the second expansion member comprises a reservoir for the treatment fluid.
